(19) United States
(12) Patent Application Publication
(10) Pub. No.: US 2006/0078767 A1
(43) Pub. Date: Apr. 13, 2006

(54) Odorant, liquid fuel for fuel cell and fuel cell

(76) Inventor: Tomoaki Arimura, Tokyo (JP)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/220,502

(22) Filed: Sep. 7, 2005

(30) Foreign Application Priority Data
Sep. 29, 2004 (JP) P.2004-285454

(51) Int. Cl. H01M 8/18; C07D 213/46

(52) U.S. Cl. 429/20; 546/315

(57) ABSTRACT

An odorant having a high odor diffusing rate, a high tolerable factor of dilution and a low percent adsorption is provided and has a pyridine derivative and a steric compound. A liquid fuel for a fuel cell and a fuel cell are provided and each has the odorant.

### CROSS-REFERENCE TO RELATED APPLICATION

This application is based upon and claims the benefit of priority from the prior Japanese Patent Application No. 2004-285454, filed on Sep. 29, 2004, the contents of which is incorporated herein by reference.

### BACKGROUND

1. Field

Embodiments of the invention relates to an odorant capable of notifying a person of the fact that the liquid mixed therewith is a dangerous material by offending the person with the odor of the liquid in case of the leakage of the liquid, a liquid fuel for a fuel cell containing the odorant, and a fuel cell containing the liquid fuel.

2. Description of the Related Art

A fuel gas such as natural gas, city gas, industrial gas and liquefied petroleum gas and liquid fuel such as gasoline, naphtha and kerosene give an extremely weak odor. As the simplest method for preventing disaster such as flaming, explosion and poisoning due to leakage of these fuel gases there has been heretofore practiced the incorporation of an odorant having a specific odor in these fuel gases for the purpose of allowing the leakage of the fuel gas to be easily perceived by human nose. As the odorants to be incorporated in the aforementioned fuels there have been heretofore used mercaptanes and sulfides. These mercaptanes and sulfides are sulfur compounds that generate sulfite gas or the like during the combustion of the fuel to disadvantage.

On the other hand, as the fuel to be used in the fuel cell there is used hydrogen gas or an alcohol-based gas such as methanol. It is thought that these fuels for fuel cells, too, should be prevented from causing disaster such as flaming, explosion and poisoning due to leakage.

For example, JP-A-2003-155488 discloses an odorant to be incorporated in hydrogen fuels for fuel cells. JP-A-2002-60766 discloses the use of an ether, ester or rose oxide having a specific structure as a fuel odorant for fuel cells. On the other hand, JP-A-2003-327982 discloses a fuel odorant obtained by incorporating at least one of ethylidene cyclohexane having a specific structure which stays liquid at 20° C. and hydrocarbon derivatives thereof and tetrahydroindene having a specific structure which stays liquid at 20° C. and hydrocarbon derivatives thereof in a fuel having a boiling point of 300° C. or less and a melting point of 20° C. or less.

However, the odorant disclosed in JP-A-2002-60766 can poison the catalyst contained in the anode or cathode of the fuel cell. On the other hand, the odorants disclosed in JP-A-2003-155488 and JP-A-2003-327982 leave something to be desired in odor diffusing rate.

JP-A-2001-214179 discloses that hydrogen, which is a fuel, can be efficiently produced by reforming a fuel oil having a real calorific value of 33,000 J/cm³ per volume and a carbon/hydrogen molar ratio of 0.52 or less. As an example of this fuel oil there is disclosed adamanthane.

### BRIEF DESCRIPTION OF THE DRAWINGS

The accompanying drawings, which are incorporated in and constitute a part of the specification, illustrate presently exemplary embodiments of the invention, and together with the general description given above and the detailed description of the exemplary embodiments given below, serve to explain the principles of the invention.

FIG. 1 is a diagrammatic view illustrating a direct methanol type fuel cell which is an illustrative, non-limiting embodiment of the fuel cell according to the invention; and

FIG. 2 is a characteristic curve illustrating the current-voltage characteristics of the direct methanol type fuel cells of Examples 1 to 6 and Comparative Example.

### DETAILED DESCRIPTION

The inventors made extensive studies. As a result, it was found that when an odorant comprising a pyridine derivative and a steric compound in admixture is used even in a small amount, a sufficient odor can be obtained and the percent adsorption of odorant to the piping, vessel, etc. can be reduced. The invention has thus been worked out.

In other words, a mixture of a pyridine derivative and a C₅-C₂₀ hydrocarbon compound or a derivative thereof, the hydrocarbon compound having a stereostructure including a plane defined by four carbon atoms and at least one carbon atom which is not contained in the plane, exhibits a high volatility and hence an enhanced odor diffusing rate.

Exemplary examples of the pyridine derivative include a pyridine derivative having a structure represented by the following formula (1). A mixture including the pyridine derivative of the formula (1) and a steric compound also gives a strong odor that is definitely different from ordinary odors due to synergism of the functional groups R1, R2 and R3 of the formula (1), a pyridine ring of the pyridine derivative and the steric compound, and thus can give a sufficient odor even when diluted by a high factor.

The piping, vessel or other devices for transporting or receiving odorants are normally made of a metal such as SUS or a resin (e.g., fluororesin, silicon resin) and thus have a fine surface roughness and a slight electrostatic charge. The aforementioned mixture has a less maldistribution of electric charge and a smaller molecular size and thus has a less adsorption to the piping, vessel or the like, making it possible to reduce the transportation loss.

It was also found that the use of an odorant including the aforementioned mixture as an odorant to be incorporated in a liquid fuel for a fuel cell makes it possible to reduce the poisoning of electrode catalyst while realizing both higher odor diffusing rate and lower percent adsorption at a high factor of dilution. Accordingly, the use of the odorant of the invention in a fuel cell makes it possible to make remarkable notice of a danger of leakage of liquid fuel without impairing the electricity-generating properties. 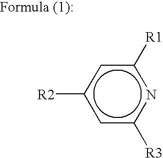
wherein R1 represents a functional group containing a sulfur atom; R2 represents an acidic functional group; and R3 represents a basic functional group. The position of the functional groups R1, R2 and R3 are not limited to those defined above. For example, R1 and R2 may replace each other.

The pyridine derivative of the formula (1) and the steric compound will be further described hereinafter.

(Pyridine Derivative of the Formula (1))

The functional group R¹ contains a sulfur atom. In order to obtain a strongly offensive odor, R1 is preferably a functional group containing a thiol group. The number of carbon atoms in the functional group R1 is preferably 12 or less (including 0). This is because when the number of carbon atoms in the functional group R1 is 12 or less, the resulting pyridine derivative has a smaller molecular size and the percent adsorption of the odorant to the piping, vessel or the like can decrease.

The functional group R2 is an acidic functional group. In order to provide a high odor diffusing rate even at a high factor of dilution, R2 is preferably an acidic functional group containing at least one selected from the group consisting of a carboxyl group, a sulfonic acid group and a phosphoric acid group.

The number of carbon atoms in the functional group R2 containing carboxyl group is preferably from 1 to 6. This is because when the number of carbon atoms in the functional group R2 containing carboxyl group is 6 or less, the resulting pyridine derivative has a smaller molecular size and the percent adsorption of the odorant to the piping, vessel or the like can decrease. The number of carbon atoms in the functional group R2 containing carboxyl group is more preferably from 1 to 3.

The functional group R3 is a basic functional group. In order to provide a high odor diffusing rate even at a high factor of dilution, R3 is preferably a basic functional group containing amino group.

Examples of the basic functional group R3 containing an amino group include —NH₂, an aliphatic amino group, and an aromatic amino group. Any of primary, secondary and tertiary amino groups may be used.

The number of carbon atoms in the basic functional group R³ is preferably 10 or less (including 0). This is because when the number of carbon atoms in the basic functional group R³ is 10 or less, the resulting pyridine derivative has a smaller molecular size and the percent adsorption of the odorant to the piping, vessel or the like can decrease.

The pyridine derivative essentially has a small maldistribution of electric charge because it has an acidic functional group R2 and a basic functional group R3. Further, when the acidic functional group R2 is a carboxyl group and the basic functional group R3 is an NH₂ group, a sufficient effect of neutralizing electric charge can be exerted. In the combination of these functional groups R2 and R3, the functional group R1 can be an SH group to obtain a pyridine derivative having a reduced molecular size and hence a lower percent adsorption to the piping or the like.

(Steric Compound)

The steric compound is a C₅-C₂₀ hydrocarbon compound R4 or derivative thereof, the hydrocarbon compound R4 having a stereostructure including a plane defined by four carbon atoms and at least one carbon atom which is not contained in the plane. The dissolution of such a steric compound in the aforementioned pyridine derivative makes it possible to give a strong odor having a high specificity even at a high factor of dilution.

When the number of carbon atoms in the hydrocarbon compound R⁴ is less than 5, the aforementioned stereostructure cannot be attained. On the other hand, a steric compound having 20 or less carbon atoms has a smaller molecular size and the resulting odorant can have a less percent adsorption to the piping or the like. Further, the resulting steric compound has a higher solubility in the pyridine derivative and has a higher odor diffusing rate or tolerable factor of dilution. The number of carbon atoms in the hydrocarbon compound R⁴ is more preferably from 8 to 14.

The derivative of the hydrocarbon compound R4 preferably has a structure represented by the following formula (A):
R4-R5

The substituent R5 is preferably a functional group having 6 or less (including 0) carbon atoms. This is because when the number of carbon atoms in the functional group R⁵ is 6 or less, the resulting steric compound has a smaller molecular size and the percent adsorption of the odorant to the piping or the like can decrease. Further, the resulting steric compound has a higher solubility in the pyridine derivative and has a higher odor diffusing rate or tolerable factor of dilution. The number of carbon atoms in the group R⁵ is more preferably from 1 to 3.

Specific examples of the steric compound include alicyclic hydrocarbons such as adamanthane, and derivatives thereof. Among these steric compounds, adamanthane or derivatives thereof have a sublimability and hence a high effect of enhancing the odor diffusing rate. Due to synergism with a pyridine derivative wherein the functional groups R¹, R² and R³ are an SH group, a carboxyl group and NH₂, respectively, adamanthane and derivatives thereof can give a specific odor and further reduce the percent adsorption of the odorant to the piping or the like. The structure of adamanthane is represented by the formula (2):

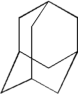

The mixing ratio of the pyridine derivative to the steric compound (pyridine derivative P:steric compound T) is preferably from 40:60 to 60:40 by weight. This is because when the mixing ratio (P:T) falls within the above defined range, a sufficient effect can be exerted. The mixing ratio (P:T) is more preferably from 45:55 to 55:45.

A solution of the steric compound in the pyridine derivative may be used as an odorant. However, a solution of the pyridine derivative and the steric compound in an organic solvent may be used as an odorant. As such an organic solvent there may be used a halogenated hydrocarbon such as dichloromethane and dichloroethane.

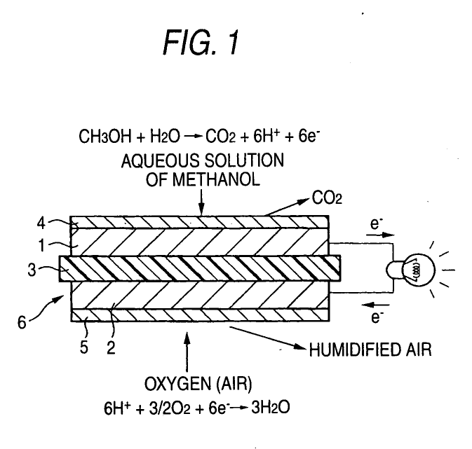

Exemplary examples of the use of odorant include a liquid fuel for a fuel cell. An example of the fuel cell using a liquid fuel is a direct methanol type fuel cell. A direct methanol type fuel cell includes an anode containing an anode catalyst layer, a cathode containing a cathode catalyst layer, and a solid electrolyte membrane between the anode and the cathode. As a liquid fuel to be supplied into the anode there is used one containing methanol. On the other hand, as an oxidizing agent to be supplied into the cathode there is used air. This direct methanol type fuel cell is diagrammatically shown in FIG. 1.

The direct methanol type fuel cell includes an anode catalyst layer 1, a cathode catalyst layer 2, a solid electrolyte membrane 3 between the anode catalyst layer 1 and the cathode catalyst layer 2, an anode diffusion layer 4 disposed on the surface of the anode catalyst layer 1 opposite the solid electrolyte membrane 3, and a cathode diffusion layer 5 disposed on the surface of the cathode catalyst layer 2 opposite the solid electrolyte membrane 3. The layered product of the five layers is generally called membrane-electrode assembly (MEA) 6.

Examples of the anode catalyst to be incorporated in the anode catalyst layer 1 include platinum alloys such as Pt—Ru alloy. On the other hand, examples of the cathode catalyst to be incorporated in the cathode catalyst layer 2 include Pt. The anode diffusion layer 4 is adapted to diffuse the liquid fuel uniformly in the anode catalyst layer 1 and is formed by, e.g., carbon paper. The cathode diffusion layer 5 is adapted to diffuse the oxidizing agent uniformly in the cathode catalyst layer 2 and is formed by, e.g., carbon paper. As the solid electrolyte membrane 3 there is used a proton-conductive polymer such as perfluoroalkyl sulfonic acid membrane.

A liquid fuel including, e.g., aqueous solution of methanol is supplied into the anode catalyst layer 1 through the anode diffusion layer 4. An oxidizing agent such as air is supplied into the cathode catalyst layer 2 through the cathode diffusion layer 5. In the anode catalyst layer 1, a reaction represented by the following reaction formula (1) occurs.
CH₃OH+H₂O→CO₂+6H⁺+6e⁻ (1)

The proton thus produced is supplied into the cathode catalyst layer 2 through the solid electrolyte membrane 3. On the other hand, the electron is supplied into the cathode catalyst layer 2 through an external circuit. In this manner, a reaction represented by the following reaction formula (2), i.e., electricity-generating reaction occurs in the cathode catalyst layer 2.
6H⁺+3/2O₂+6e⁻→3H₂O (2)

Carbon dioxide and water produced by the foregoing electricity-generating reaction are discharged to the exterior. The excessive methanol which has not been consumed in the anode catalyst layer 1 can be recovered and reused as a fuel.

The incorporation of the odorant of the invention in the aforementioned liquid fuel makes it possible to make remarkable notice of danger because the odorant of the invention can be diffused at a high rate even when diluted at a high factor. Since the odor thus given is definitely different from ordinary odors, persons in the vicinity of the fuel cell can be sufficiently informed of danger. Further, since the odorant of the invention has a low poisoning effect on the anode catalyst and cathode catalyst, the electricity-generating efficiency of the fuel cell cannot be impaired even when the odorant of the invention is incorporated in the liquid fuel. Moreover, since the odorant of the invention exhibits a low percent adsorption to the liquid fuel tank or piping, the drop of the effect of the odorant from before reuse can be inhibited when excess methanol is recovered and reused as a fuel.

The concentration of the odorant in the liquid fuel is preferably 10% by weight or less. This is because when the concentration of the odorant exceeds 10% by weight, it is likely that the electricity-generating efficiency of the fuel cell can be deteriorated. The concentration of the odorant in the liquid fuel is more preferably 1% by weight or less, even more preferably 0.5% by weight or less. In order to fully exert the effect of the odorant, the concentration of the odorant in the liquid fuel is preferably 0.001% by weight or more.

The invention will be further described hereinafter in connection with the aforementioned drawings.

### EXAMPLE 1

An odorant composed of 2-thiol-4-carboxy-5-amine as a pyridine derivative and adamanthane as a steric compound was prepared. The pyridine skeleton of 2-thiol-4-carboxy-5-amine is represented by the aforementioned formula (1). The substituents R¹, R² and R³ on the pyridine skeleton are represented by the structural formulae shown later. The structural formula of methyl adamanthane is also shown later.

2-thiol-4-carboxy-5-amine and methyl adamanthane were added to dichloromethane to obtain a dichloromethane solution of odorant. The content of 2-thiol-4-carboxy-5-amine and the content of methyl adamanthane in the dichloromethane solution are 20% and 10% by weight, respectively. The mixing ratio of the pyridine derivative to the steric compound (by weight) is set forth in Table 1 below.

### EXAMPLES 2 TO 6

The pyridine skeleton represented by the aforementioned formula (1), the pyridine derivative having substituents R¹, R² and R³ represented by the structural formulae shown later and the steric compound having a structure represented by the structural formulae shown later were added to dichloromethane at a mixing ratio set forth in Table 1 below to obtain a dichloromethane solution of odorant. COMPARATIVE EXAMPLE

As an odorant there was prepared dimethyl sulfide.

<Measurement of Odorant Diffusing Rate Ratio>

The comparative odorant was diluted with 100 ml of a 3% aqueous solution of methanol using a syringe to attain a content of 0.1% by weight. The solution was then stirred at 300 rpm using a magnetic stirrer and a teflon (trade name) agitator for 10 minutes.

A glass tube having an inner diameter of 10 cm and a length of 1 m was then placed on a horizontal table. A person who had been chosen as a monitor brought his or her nose close to one end of the glass tube. 100 microlitter of the diluted odorant solution was sampled through a microsyringe from which it was then injected into the glass tube at the other end thereof. The time at which the injection of the sample begins was defined as 0 second. The time required until the monitor feels the odor was defined as T1 second from which the diffusing rate T1 (m/sec) was then calculated. This procedure was repeatedly effected three times. The measurements were then averaged to determine T1av.

The dichloromethane solutions of odorants of Examples 1 to 6 were each diluted with an aqueous solution of methanol, and then measured for diffusing rate (m/sec) from which T2av was then determined in the same manner as mentioned above. From T1av/T2av was then calculated the diffusing rate ratio. The measurements are set forth in Table 1 below.

<Measurement of Maximum Factor of Dilution>

The comparative odorant was diluted with a 3% aqueous solution of methanol by a factor of 10, 100, 500, 1,000, 2,000 and 5,000 using a graduated flask. The six diluted solutions thus obtained were each transferred into a beaker over which the monitor then smelled the odor to see if the odor was perceivable. The maximum factor of dilution at which the odor can be perceived was defined as D1.

The dichloromethane solution of the odorants of Examples 1 to 6 were each determined for maximum factor of dilution D2 at which the odor can be perceived in the same manner as mentioned above. From D2/D2 was calculated the maximum factor of dilution. The results are set forth in Table 1 below.

<Measurement of Adsorption Concentration>

The comparative odorant was diluted with a 3% aqueous solution of methanol to attain a content of 0.1% by weight. 20 ml of the diluted solution was injected into a PFA tube having an inner diameter of 8 mm and a length of 10 m including a liquid pump connected thereto at a point along the length thereof. The diluted solution was then circulated through the PFA tube for 3 hours. Thereafter, the solution in the tube was driven out of the tube by an air pump so that it was collected. The solution thus collected was washed with 50 ml of purified water. The wash water and the aqueous solution of methanol with odorant which had been initially sampled were combined to make 100 ml. The aqueous solution was then analyzed by a high speed liquid chromatography to determine the concentration of dimethyl sulfide (microgram/ml). Thus, adsorption concentration C1 was obtained.

The dichloromethane solution of odorants of Examples 1 to 6 were each measured for total adsorption concentration C2 of pyridine derivative and steric compound in the same manner as mentioned above. From C2/C1 was then calculated adsorption concentration ratio. The results are set forth in Table 1 below.

TABLE 1

| | Pyridine derivative | | | |
|---|---|---|---|---|
| Example | R¹ | R² | R³ | Steric compound (T) |
| 1 | SH | COOH | NH₂ | 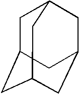 |
| 2 | SH | SO₃H | NH₂ | 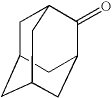 |
| 3 | SH | PO₄H | NH₂ | 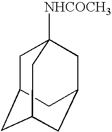 |
| 4 | SH | COOH | NH₂ | 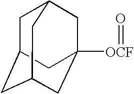 |
| 5 | CH₂COSH | CH₂COOH | 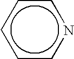 | 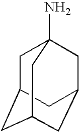 |
| 6 | CH₂COSH | CH₂COOH | 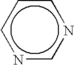 | 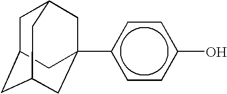 |

| | % Mixing ratio | Diffusing | Maximum factor | Adsorption |
|---|---|---|---|---|
| | (P:T) | rate ratio | of dilution | concentration ratio |
| Example 1 | 50:50 | 1.1 | 1.2 | 1.1 |
| Example 2 | 55:45 | 1.1 | 1.1 | 1.2 |
| Example 3 | 40:60 | 1.1 | 1.2 | 1.2 |
| Example 4 | 55:45 | 1.2 | 1.1 | 1.1 |
| Example 5 | 50:50 | 1.2 | 1.1 | 1.2 |
| Example 6 | 60:40 | 1.1 | 1.1 | 1.2 |
| Comparative | — | 1 | 1 | 1 |
| Example | | | | |

As can be seen in Table 1 above, the odorants of Examples 1 to 6 comprising a pyridine derivative and a steric compound can be diffused at a higher rate than the comparative odorant and can be perceived offensive even at a higher factor of dilution than the comparative odorant. It was also found that the odorants of Examples 1 to 6 can be adsorbed to the piping less than the comparative odorant.

The odorants of Examples 1 to 6 and the comparative odorant were each used to prepare a direct methanol type fuel cell which was then evaluated for current-voltage characteristics.

<Assembly of Single Cell>

Platinum-ruthenium was supported on a carrier made of carbon powder in an amount of 2 mg/cm² to prepare an anode catalyst. A slurry containing the anode catalyst was then spread over a carbon paper to form an anode catalyst layer thereon.

Separately, platinum was supported on a carrier made of carbon powder in an amount of 1 mg/cm² to prepare a cathode catalyst. A slurry containing the cathode catalyst was then spread over a carbon paper to form a cathode catalyst layer thereon.

The anode catalyst layer was provided on one side of a perfluoroalkylsulfonic acid membrane which is a solid electrolyte layer. The cathode catalyst layer was provided on the other side of the perfluoroalkylsulfonic acid membrane. These layers were then subjected to hot contact bonding to prepare a membrane-electrode assembly (MEA) having an electrode area of 5 cm².

The various membrane-electrode assemblies were each disposed between two sheets of carbon separator having a serpentine channel. The laminates were each disposed between two sheets of collector. The layered product was each bolted to prepare a single cell to be evaluated.

<Preparation of Methanol Fuel>

The dichloromethane solution of odorants of Examples 1 to 6 and the comparative odorant were each dissolved in an aqueous solution of methanol such that the odorant concentration reached 0.1% by weight to obtain 7 methanol fuels.

These methanol fuels were each then injected into the methanol-water tank of a device for evaluating direct methanol type fuel cell.

<Evaluation of Single Cell>

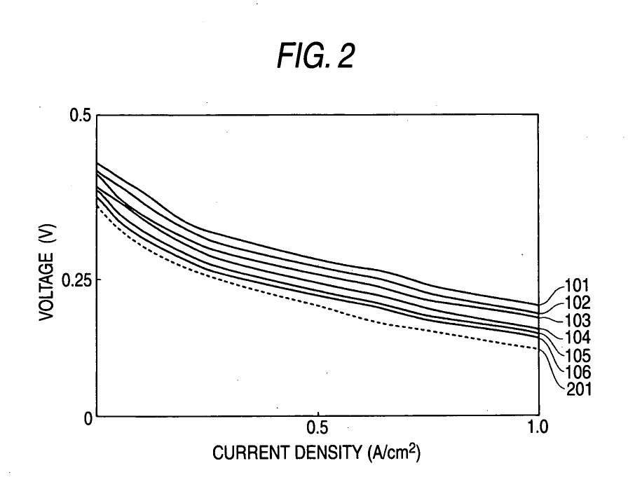

The single cell thus prepared was mounted on the device for evaluating direct methanol type fuel cell. The aforementioned aqueous solution of methanol was supplied into the single cell on the anode side thereof at a flow rate of 3 ml/min. Air was supplied into the single cell on the cathode side thereof at a flow rate of 15 ml/min. Under these conditions, the single cell was then observed for current-voltage curve at a cell temperature of 70° C. The results are shown in FIG. 2. Lines 101 to 106 represent the results of Examples 1 to 6, respectively, and line 201 represents the result of Comparative Example.

As can be seen in FIG. 2, the odorants of Examples 1 to 6 cause a less drop of output due to catalyst poisoning and thus have a less effect on the output of the fuel cell than the comparative odorant.

The invention is not limited to the aforementioned embodiments. In the implementation of the invention, the constitutions may be changed without departing from the spirit of the invention. Further, various inventions may be worked out by properly combining a plurality of constitutions disclosed in the aforementioned embodiments. For example, some of all the constitutions disclosed in the embodiments may be deleted. Moreover, constitutions selected from different embodiments may be properly combined.

What is claimed is:

1. An odorant comprising:
a pyridine derivative; and
a steric compound,
wherein the steric compound is a hydrocarbon compound having 5 to 10 carbon atoms or a derivative thereof, and the hydrocarbon compound has a stereostructure comprising a plane defined by four carbon atoms and at least one carbon atom that is not contained in the plane.

2. The odorant as defined in claim 1, wherein the pyridine derivative has a functional group containing a sulfur atom, an acidic functional group and a basic functional group.

3. The odorant as defined in claim 1, wherein the pyridine derivative has a structure represented by formula (1): 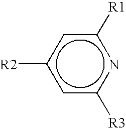
wherein R¹ represents a functional group containing a sulfur atom; R² represents an acidic functional group; and R³ represents a basic functional group.

4. The odorant as defined in claim 3, wherein R¹ is a functional group containing a thiol group.

5. The odorant as defined in claim 3, wherein R² is an acidic functional group containing at least one selected from the group consisting of a carboxyl group, a sulfonic acid group and a phosphoric acid group.

6. The odorant as defined in claim 5, wherein the acidic functional group has a carboxyl group and has 1 to 6 carbon atoms.

7. The odorant as defined in claim 6, wherein the acidic functional group has 1 to 3 carbon atoms.

8. The odorant as defined in claim 3, wherein R³ is a basic functional group containing an amino group.

9. The odorant as defined in claim 1, wherein the hydrocarbon compound has 8 to 14 carbon atoms.

10. The odorant as defined in claim 1, wherein the derivative of the hydrocarbon compound has a functional group having 6 or less carbon atoms.

11. The odorant as defined in claim 10, wherein the functional group in the derivative of the hydrocarbon compound has 1 to 3 carbon atoms.

12. The odorant as defined in claim 1, wherein the steric compound is adamanthane or a derivative thereof.

13. The odorant as defined in claim 1, which has a mixing ratio of the pyridine derivative to the steric compound of from 40:60 to 60:40 by weight.

14. A liquid fuel for a fuel cell, comprising: a liquid fuel; and an odorant dissolved in the liquid fuel.

15. The liquid fuel for the fuel cell as defined in claim 14, wherein the odorant comprises a pyridine derivative and a steric compound.

16. The liquid fuel for the fuel cell as defined in claim 14, which has a concentration of the odorant in the liquid fuel of 10% by weight or less.

17. The liquid fuel for the fuel cell as defined in claim 14, wherein the liquid fuel contains methanol.

18. A fuel cell comprising:
an anode;
a cathode;
a solid electrolyte membrane between the anode and the cathode; and
a liquid fuel to be supplied into the anode, the liquid fuel containing an odorant.

19. The fuel cell as defined in claim 18, wherein the odorant comprises a pyridine derivative and a steric compound.
